(19) United States
(12) Patent Application Publication
(10) Pub. No.: US 2026/0355075 A1
(43) Pub. Date: Nov. 12, 2026

(54) CHLORINE-MODERATING FOAM, CREAM AND GEL COMPOSITIONS FOR USE ON THE HUMAN BODY AND HAIR

(71) Applicant: Jonathan Foss, Waconia, MN (US)

(72) Inventor: Jonathan Foss, Waconia, MN (US)

(21) Appl. No.: 19/202,050

(22) Filed: May 8, 2025

(51) Int. Cl. A61K 8/04; A61Q 5/02; A61Q 19/00

(52) U.S. Cl. CPC A61K 8/046; A61Q 5/02; A61Q 19/00; A61K 2800/30; A61K 2800/87

(57) ABSTRACT

A method of providing a stable aqueous composition from an aerosol dispenser for dechlorinating skin, especially dechlorinating skin, hair and/or nails of humans. The aqueous composition includes at least water and an oil-soluble bis-ascorbate in a weight ratio of about 0.1 to 25% by weight of the total aqueous composition. The composition may be free of ascorbic acid, and the oil-soluble ascorbate is a bis or tri- or tetraalkanol ascorbate such as bisglyceryl ascorbate. A narrow range of specific classes of relatively inert (towards ascorbates) propellants is essential to the practice of the present invention.

### BACKGROUND OF THE INVENTION

### 1. Field of the Invention

The disclosure relates to aerosol delivery of foam, cream and gel compositions and delivery methods for decreasing or eliminating adverse effects of chlorine on the human body, including the skin and hair.

### BACKGROUND OF THE ART

Humans contact with chlorine in a number of different ways. For example, swimming is a popular form of exercise and pastime. By its nature, swimming requires immersing oneself in a body of water. People may swim in either natural bodies of water (such as lakes, oceans, rivers, etc.), or man-made swimming pools.

Man-made swimming pools (including traditional swimming pools, hot tubs, municipal water supplies, etc., collectively referred to generally herein as “pools”) are usually smaller than naturally occurring bodies of water. They are also usually self-contained structures, consisting of a finite body of water separated from the surrounding environment, for example by walls. Pools provide an aqueous environment that is kept within a biologically habitable temperature range, such as about 65-90 °F with a Free Chlorine residual between 1 and 5 ppm and a pH between 7.2 and 7.8. Some pools may be kept cooler or warmer. For example, a hot tub may be maintained at more than 95 °F but typically use bromine as Hypochlorous Acid is increasingly ineffective at higher temperatures.

A swimmer brings a variety of living and non-living substances into the pool. For example, the swimmer's skin, hair, saliva, urine, sweat, and other secretions may come into contact with the pool water. Owing to the aqueous medium and adequate temperature, pools provide a suitable environment for living organisms, such as bacteria, to thrive.

Pools are usually treated with oxidizing chemicals chosen to prevent the growth of harmful organisms, such as bacteria and parasites like E. coli and Cryptosporidium. Properly used, these chemicals keep the pool water substantially free from harmful contaminants. For example, many pools are treated with oxidizing agents (e.g., chlorine, hypochlorite salts such as calcium hypochlorite or sodium hypochlorite, hypochlorous acid) or to a much lesser extent, brominating or iodating agents (e.g., bromine, salts comprising bromine, iodine, etc.). These Halogens sanitize the water at low levels and serve as oxidizers at higher levels.

The chemicals used to treat pool water work by reacting with certain molecules that come into contact with the pool water. For example, these chemicals may react with bacteria's biologically important molecules, thereby killing the bacteria.

In addition to reacting with bacteria and other contaminants, however, some pool chemicals react with elements of the swimmer, such as the swimmer's body, the swimmers' hair and/or the swimmer's attire. For example, the fibers that make up the exterior of the body, such as skin, hair, eyes, and nails (collectively referred to herein as “exterior body elements” of “body fibers,” and intending to include keratinous fibers that make up the hair, skin, and nails, as well as mucous membranes), comprise proteins. By way of example, human hair is made largely from alpha keratin (alpha-keratin). Those proteins are made from amino acids. All amino acids, including those making up proteins, have one or more N—H bonds. In alpha-keratin, the most abundant amino acid is cystine, which accounts for about 15% of the protein. Monomeric L-cystine has two N—H bonds. The oxidized dimmer of cystine has four N—H bonds. When present within a protein, each cystine residue has one N—H bond.

The N—H bonds in the amino acids in body fibers can react with the chemicals found in pool water. For example, one or more N—H bonds in an amino acid in the protein of hair or skin can react with a chlorinating agent used in pool water to form N—Cl, an amino chloride. Notably, the reacted amino acid, now containing an N—Cl bond, is still part of the protein in the hair or skin.

Although a swimmer can rinse the residual pool water from his/her skin, hair, attire, etc., after swimming, such rinsing will not effectively eliminate all the adverse effects of exposure to the chemically-treated pool water. Traditional soaps are ineffective at breaking the millions of N—CL bonds that form on the skin and hair. For example, where the body's proteins have chemically reacted with the pool chemicals, they are physically changed but, at least in part, remain part of the body, i.e., they are not all rinsed away such as during normal showering.

Those remaining pool chemicals can be released throughout the day, for example as a result of exposing the skin, hair, etc., to moisture, e.g., water. As discussed above, after swimming in a pool with a chlorinating and/or brominating agent, proteins of the human body may become chlorinated and/or brominated. Subsequent exposure of those chlorinated and/or brominated amino groups to water (e.g. rain or sweat) may release these volatile chemicals, which may be corrosive or irritating. Certain skin conditioning agents have reacted with chlorine chemicals on the skin and can even cause extreme reactions. One skin moisturizer when applied to dry skin after swimming in a chlorinated pool reacted badly, turning the skin of the swimmer “red as a lobster” and caused intense burning pain as it reacted with the chlorine compounds. The particular conditioner plus dried Hypochlorous acid N—CL bonds didn't mix well. The previous composition was used and repeated this experiment many times, just in very small areas, and skin always reacted the same.

Some of these corrosive molecules may be harmful to body and/or textile fibers (e.g. clothing such as swimming attire), or may cause an unpleasant sensation upon contact with the body and/or clothing fibers. Additionally, some of the volatile molecules may be perceived by the nose when liberated from the body and/or clothing, giving rise to odors. These odors are commonly referred to as simply “chlorine” or “pool odor,” and are considered a more chemical odor, rather than the type of odor naturally produced by the body. Swimmers post workout can be smelled (wreaking) From 12 feet away. Chorine is odorless, but incredibly pungent as a mono- di- or tri-chloramine.

Also, when pool chemicals, such as chlorine or bromine, react with the biological molecules forming the skin and/or eyes, those reactions may cause irritation. For example, some swimmers report itchy or inflamed skin following swimming in pools. Some swimmers indicate that mucous membranes, such as the sensitive nasal skin, become itchy and irritated following swimming.

Although some people have reported liking “pool odor,” as reminiscent of the pleasures associated with swimming itself, most people do not like pool odor or, if strong enough, find the odor irritating, such as to the eyes and lungs. Additionally, since the chemicals liberated may irritate the skin and/or damage the hair, many people wish to prevent “pool odor” and/or the symptoms associated with it. Additionally, green hair, shiny hair and loss of body hair are secondary problems associated with the N—CL reaction with human skin and hair.

As discussed above, rinsing or washing, e.g. body fibers, does little to eliminate pool odor and/or skin irritation. Mitigating (i.e. decreasing to some extent or eliminating entirely) the effects of exposure to chlorinating and/or brominating agents requires reversing the chemical reaction between those chemicals and the proteins making up the human body. This requires converting the amino-halide bonds, e.g., chloramine (N—Cl) and/or bromamine (N—Br) groups into amino (N—H) groups. However, the soaps, shampoos, and conditioners currently known do not effectively convert N—Cl and/or N—Br bonds on body fibers back into N—H bonds. Accordingly, N—Cl and/or N—Br remain bonded to the body fibers following rinsing, washing, shampooing, and/or conditioning the body fibers, such as the skin and/or hair.

Some known shampoo and soap formulations are directed to mitigating the effects of exposure to chlorinating and/or brominating agents. For example, U.S. Pat. No. 4,295,985 discloses “a method of removal of chlorine retained by human skin and hair after exposure to chlorinated water, and soap and shampoo compositions adapted to effect said removal.” That patent teaches applying urea and thiosulfate salts to the hair and/or body following exposure to chlorinating agents.

Other known formulations have sought to remove minerals from hair in an effort to prevent discoloration of the hair. For example, U.S. Pat. No. 5,804,172 discloses compositions aimed at removing mineral deposits from hair exposed to hard water, particularly the calcium, magnesium, iron, and copper present in some municipal water sources. That patent discloses compositions including four ingredients, which are said to remove minerals from the hair due to the “synergistic combination” of ingredients. Within those compositions, a reducing agent, such as ascorbic acid, is included in an amount chosen to reduce oxidized cysteine-iron bonds. The patent discloses four-component compositions comprising 2.1 percent w/w of ascorbic acid, which is said to be sufficient to reduce the oxidation state of iron ions bonded to hair.

Additional known formulations have sought to remove chlorine from hair by treating the hair with ammonium lauryl sulfate, cocamide diethanolamine, sodium bicarbonate, cocobetaine, and water. See U.S. Pat. No. 4,547,364.

Finally, a host of other formulations promise to treat damaged hair and/or skin following exposure to swimming pools by using various combinations of ingredients. For example, U.S. Pat. No. 4,690,818 discloses a combination of hair and skin conditioners and moisturizers, namely, “a combination of coco-diammonium hydrolyzed keratin and a mixture of monosaccharides and disaccharides . . . .”

U.S. Pat. No. 9,302,909 (Chadeayne) discloses methods of mitigating the effects of exposure to oxidizing agents, chlorinating and/or brominating agents on biological and/or synthetic fibers, by treating the body and/or clothing fibers with an effective amount of a composition comprising compounds of formula A, which include ascorbic acid and sodium salts of ascorbic acid (sodium ascorbate). Also disclosed are compositions comprising compounds of formula A, for use in treating biological and/or synthetic fibers to mitigate the effects of exposure to chlorinating and/or brominating agents.

U.S. Pat. No. 4,295,985 discloses a method of removal of chlorine retained by human skin and hair after exposure to chlorinated water, and soap and shampoo compositions adapted to effect said removal by removing rapidly chlorine retained by the skin and hair of, for example, swimmers, after exposure to chlorinated water by means of a stoichiometric excess of a suitable reducing agent, such as an alkali thiosulfate and the like in aqueous solution, and with toilet soaps and shampoos comprising said agent adapted to effect said removal.

U.S. Pat. No. 7,244,453 (Lucia M. Litman) discloses that retention of chlorine and hypochlorite in hair is reduced by treating the hair with a defined composition, allowing the composition to remain on the hair for at least 10 seconds, and then rinsing off the composition with water. The composition comprises at least 1% by total weight of the composition of potato particles having an average diameter of less than 10 microns when dried; at least 1% by total weight of the composition of clathrate; at least 1% by weight of surfactant; and water.

Alternative and more effective treatments for the effects of pool chemicals and especially chlorine chemistry are needed. Each of these inventions have fatal flaws not allowing them to be truly effective. Thiosulfates and other formulas react too slowly, Ascorbic acid is not shelf-stabile, oxidizes and turns brown and loses effectiveness.

### SUMMARY OF THE INVENTION

An aerosol delivery system is used in a method for the delivery of an aqueous composition comprising water and an oil-soluble bis-ascorbate in a weight ratio of about 0.1 to 25% by weight of the total aqueous composition, wherein the system comprises a) a housing, an actuator, an aqueous composition delivery stem, a nozzle, and a dip tube extending into a storage compartment within the housing, and further wherein the storage compartment is defined as oxygen-free and contains the aqueous composition and a propellant selected from the pH-influencing acceptable range (does not reduce the pH of the solution to less than 5.5) and especially when selected from the group consisting of hydrofluoroolefins, nitrogen, and nitrogen oxides,

Aqueous solutions, suspensions and dispersions comprising water and a specific class of stabilized ascorbates, especially oil-soluble ascorbates such as bisascorbates such as bis-alkyl ascorbates (e.g., tetrahexyldecyl Ascorbate) and other substituted bis ascorbate compounds (and particularly bisglyceryl ascorbate) and tetraalkyl ascorbates (and with substituted alkyl groups) and bis-aryl ascorbates and other aryl ascorbate. A method of dechlorinating skin, especially dechlorinating skin, hair and/or nails of humans comprising contacting said skin, hair and/or nails with an aqueous composition for sufficient time to reduce halogen effects on the skin, hair and/or nails. The aqueous composition includes at least water and an oil-soluble bis-ascorbate in a weight ratio of about 0.1 to 25% by weight of the total aqueous composition. The composition may be free of ascorbic acid (e.g., less than 5% ascorbic acid to total ascorbate), and the oil-soluble ascorbate is a bis- or tri- or tetra-alkanol ascorbate such as bis-glyceryl ascorbate. There are a variety of delivery methods for this stabile solution depending upon the user's situation.

### DESCRIPTION OF THE FIGURES

FIG. 1 shows a schematic of a generic aerosol delivery can,

### DETAILED DESCRIPTION OF THE INVENTION

An aerosol delivery system is used in a method for the delivery of an aqueous composition comprising water and an oil-soluble bis-ascorbate in a weight ratio of about 0.1 to 25% by weight of the total aqueous composition, wherein the system comprises a) a housing, an actuator, an aqueous composition delivery stem, a nozzle, and a dip tube extending into a storage compartment within the housing, and further wherein the storage compartment has less than) 0.001% by volume of free oxygen and contains the aqueous composition and a propellant selected from the group consisting of hydrofluoroolefins, nitrogen, and nitrogen oxides.

Considering propellants and other ingredients as being non-oxidants or oxidizers is important in selecting ingredients. For example, in direct additions of chemistry to pools, sodium thiosulfate and potassium thiosulfate work well at reducing high levels of chlorine. But when put into a shampoo, they become ineffective, and are not able to remove chlorine from pool water. It is likely that the sodium and potassium thiosulfates react to ingredients in solution. This explains why 25 Swim shampoos on the market fail our laboratory testing for removal of chlorine from skin, nails and hair.

Similarly, the ingredients choices are critical, the pressure and propellant choices as well, and method of application foam, styling mousse, shampoo gel, conditioner lotion, c-serum lotion, detangler spray, sunscreen with vitamin C, Aloe (w/ vitamin C) and hand soap.

Aqueous solutions comprising a specific generic class of stabilized ascorbates are used in a wide range of care products for humans and mammals to mediate effects from halogen contaminants on skin, especially chlorine deposits and reactants on humans from exposure to chlorinated water. The generic class of ascorbates especially includes oil-soluble ascorbates, such as bisascorbates such as bis-alkyl ascorbates (e.g., tetrahexyldecyl Ascorbate) and other substituted bisacompounds (and particularly bisglyceryl ascorbate) and tetraalkyl ascorbates (and with substituted alkyl groups) with a total of at least 10 carbon atoms per alkyl substituent and from 2 up to 14 carbon atoms per alkyl) and bis-aryl ascorbates and other aryl ascorbates with at least a total of 10 combined total carbon atoms in all of the aryl groups.

The structural formula of bisglyceryl ascorbate is (as a salt, such as a sodium salt thereof), which salts are included within the definition of ascorbates:

This is the structure of 3-Glyceryl Ascorbate, and all glyceryl ascorbates are included in the practice of the present invention.

The liquid or gel compositions may be carried on a sheet or film of material, such as natural or synthetic carriers. Cloth, wool, cellulosic sheets, porous sheets, superabsorbent polymer sheets (such as those disclosed in U.S. Pat. Nos. 10,182,946 and 9,901,128 (Gray) and blends of materials can be used to carry the aqueous solutions. “Wet-wipes” with a stabile anti-oxidant is one embodiment for the application of this invention. These can then be used to wipe on the skin to remove chlorine chemicals. In the case of the superabsorbent materials, these can often be reactivated numerous times by application of moisture to partially exhausted sheets and used multiple times, although the solutions will eventually become completely leached from the carrier.

The ascorbates may have Na, Li, Ca, K, NH4 and other cations as the salt alternatives. The simplest solution useful in the practice of the present technology would be water and the water-dispersible to water-soluble ascorbates described in the practice of the present invention. Additives typical for use in shampoos, skin cleansers, skin washes, hand washes and soaks can then be added to the basic formulation to make specific classes of products. Shampoo, Soaps, Body Wash, Lotions, Gels, Sprays as listed at the beginning. (is this listed at the beginning?) The final compositions will contain from about 0.1 to 25% by total weight of the composition as the oil-soluble ascorbate with the remainder of the 100% of the compositions comprising water and typical care additives that would not adversely react with the oil-soluble ascorbates. Such typical ingredients include surfactants, thickening agents, fragrances, moisturizers, anti-inflammatories, dyes, pigments, UV-absorbers, and the like.

General detergent anionic-cationic surfactant mixtures are well known to the art. See generally, U.S. Pat. Nos. 5,441,541, 5,472,455, 5,204,010, 4,790,856, 4,298,480, 3,730,912 (all to The Colgate-Palmolive Company), U.S. Pat. Nos. 5,622,925, 5,607,980, 5,565,145, 4,913,828, 4,659,802, 4,436,653, 4,338,204, 4,333,862, 4,132,680 (all to The Procter & Gamble Co.); also see WO 97/03164, WO 97/12022 and WO 96/37591 (all to The Procter & Gamble Co.), and WO 97/28238 and WO 97/15647 (both to Reckitt & Colman, Inc.). See also, U.S. Pat. Nos. 5,610,187 and 4,247,538 (both to Witco Corp.), U.S. Pat. No. 5,344,949 (to Th. Goldschmidt AG), U.S. Pat. Nos. 5,332,854 and 5,324,862 (both to Dai-Ichi Kogoyo Seiyaku Co., Ltd.), U.S. Pat. No. 4,273,760 (to National Starch and Chemical), and U.S. Pat. No. 4,264,457 (to DeSoto, Inc.).

Compositions comprising anionic-cationic surfactant mixtures are also relatively well known. U.S. Pat. No. 6,007,802 discloses a conditioning shampoo composition with excellent cleaning performance and improved levels of conditioning while minimizing any adverse effect associated with build-up; the disclosed compositions generally comprise an ethoxylated alkyl sulfate, amphoteric surfactant, insoluble, dispersed conditioning agent (nonionic, cationic silicone), synthetic esters, and cellulosic cationic polymers. U.S. Pat. No. 5,939,059 discloses a 2-in-1 conditioning shampoo comprising an anionic surfactant (alkyl sulfate or ether sulfate) and ester quats, with optional amide. U.S. Pat. No. 5,747,436 discloses a low static conditioning shampoo comprising an anionic and an amphoteric surfactant, complex acid:amine (1:1 mole ratio) and polyquaternary compound. U.S. Pat. No. 5,607,980 discloses topical compositions having improved skin feel comprising an anionic surfactant (alkyl sulfate, ether sulfate, isethionate), a cationic surfactant and an amphoteric surfactant. U.S. Pat. No. 5,997,854 discloses a conditioning shampoo formulation comprising a quaternary ammonium component, an emulsifier, an amphoteric, an alkyl polyglycoside surfactant. U.S. Pat. No. 5,145,607 discloses an optically clear conditioning shampoo comprising anionic (alkyl sulfate or alkyl ether sulfate) and cationic surfactants. U.S. Pat. No. 4,931,216 discloses detergent compositions comprising an anionic or amphoteric surface-active agents and a branched quaternary ammonium salt. U.S. Pat. No. 4,744,977 discloses quaternary ammonium compound hair conditioners in combination with an anionic surfactant. U.S. Pat. No. 5,661,189 discloses mixtures of anionic, cationic, amphoteric, nonionic, zwitterionic surfactants, along with benefit agents, thickening agents an small amounts of soap. These references are incorporated herein by reference for their general disclosure and compounding methods. While ascorbic acid is very beneficial to the skin, it is notoriously difficult to stabilize in cosmetic formulations. This is because it is such an excellent antioxidant, which means that it will readily donate electrons to the oxygen content in air. After becoming oxidized, the ascorbic acid molecule is ineffective and cannot provide skin benefits. Therefore, a product that contains ascorbic acid needs to be packaged to prevent oxidation. When purchasing a product containing ascorbic acid, make sure that the packaging is opaque and is either an airless pump bottle or a tube. (Zero contact with oxygen, inert gases such as Nitrogen)

The safety of ascorbic acid and its salts has been assessed by the Cosmetic Ingredient Review (CIR) Expert Panel. The Panel reviewed the scientific data and concluded that these ingredients were safe for use in cosmetic and personal care products. Since ascorbic acid is an acidic ingredient, some people may experience sensitivity, redness, or stinging after using products with this ingredient. These side effects typically occur when high concentrations of ascorbic acid are used, or when ascorbic acid is combined with other ingredients that exfoliate the skin. The oil-soluble ascorbates of the present invention are also more gentle on the skin and hair than ascorbic acid.

Furthermore, chlorine (including, for example, gaseous or solvated Cl₂, chlorine-comprising oxidizing agents, and salts thereof) has a multitude of uses in both household and industrial applications. For example, it can be used for disinfecting, whitening, bleaching, and clarifying materials. Chlorine is often used as an antimicrobial agent. For example, sodium hypochlorite (a chlorinating agent) is known to kill a broad array of microbes. Owing to the efficacy, cost, and versatility of chlorinating agents, they are amazingly attractive reagents for a variety of home and industrial applications. These non-pool applications can also cause similar effects to pool chlorine when the materials are spilling on persons or merely adsorbed from the air.

A down side to using chlorinating agents in the home and industry is that they can react with many of the materials to which they are exposed—often materials that the user would like to keep free from chlorination. Because materials and surfaces that are exposed to chlorine and chlorinating agents undergo a chemical reaction with the chlorine, their chemical composition becomes altered. Part of the chlorine and/or chlorinating agent becomes bound to the material or surface. Accordingly, one cannot simply wash away the residual chlorine. The bound chlorine must first be liberated before it can be washed away with traditional soaps and surfactants. The present compositions, even though designed for use on skin, hair and/or nails, may also be used to wash surfaces in an industrial setting where chlorine compounds may deposit.

In the food industry, it may be desired to chlorinate the water that is used to chill poultry carcasses, such as chicken, but chicken producers would like to avoid chlorinating the biological tissues making up the chicken. Poultry is an important part of the worldwide animal food market. The poultry industry raises chickens, kills them, and then processes them into a form that is both convenient and safe for the consumer to use in preparing meals. Converting live chickens into healthy food presents challenges to the chicken industry. In particular, poultry provides an excellent medium for the growth of microorganisms, such as Pseudomonas, Staphylococcus, Micrococcus, Acinetobacter, Moraxella, and Salmonella. Workers can easily have their hair and nails absorb these chlorine compounds of have their skin adsorbed with the compounds.

In various embodiments, salts and/or derivatives (including but not limited to oxidized forms) of ascorbates may be used in place of, or in addition to the preferred bisglycidyl ascorbate. Salts and/or derivatives of these oil-soluble compounds would function similarly within the context of the disclosed compositions and methods. As non-limiting examples, esters or ethers of the ascorbate may be used in the compositions and methods of the disclosure. Unless otherwise noted or individually recited, when the term “ascorbic acid” is used herein, it is intended to include salts (e.g. sodium, potassium, calcium, etc. of the ascorbate) and/or degradation products and/or derivatives (e.g., oxidized forms) thereof, whether or not so stated. For example, when present within an aqueous solution, the term “ascorbate” as used herein is intended to include products formed as a result of ascorbate reacting with oxygen dissolved in water and/or oxygen.

Furthermore, as known, ascorbic acid has stereogenic centers in its structure, for example the carbons modified by the (R)- and (S)-indicators in the chemical name (R)-3,4-dihydroxy-5-((S)-1,2-dihydroxyethyl)furan-2(5H)-one. This disclosure contemplates use of any combination of stereoisomers of ascorbic acid as the base of the ascorbate, including isolated stereoisomers, and all mixtures thereof.

The phrases “effective amount of ascorbic acid” or “effective amount of a composition comprising ascorbic acid”, and variations thereof, as used herein, are intended to include any amount of ascorbate (as defined herein to include salts, degradation products, and derivatives) that is sufficient to mediate or even convert any or all of the chlorine compounds (chlorine, chloride, hypochlorite, etc.), including N—Cl bonds or groups adsorbed or bound to the hair, skin or nails being treated into N—H. In at least one embodiment, an effective amount of ascorbic acid means an amount sufficient to mitigate (i.e., reduce to any degree or eliminate completely) the perceptible adverse effects of exposure to the chlorine compounds. For example, in one embodiment, an effective amount of ascorbic acid would be an amount sufficient to reduce or eliminate an odor of chlorine from hair, skin and/nails, such as, for example, to reduce or eliminate an undesirable “pool odor” present on the body and/or clothes of a swimmer. In compositional amounts, the liquid aqueous compositions may comprise from 0.05% by weight up to 25% (you used 0.1% earlier in document) or even 40% by weight of the oil-soluble ascorbate to 100 parts by weight of total aqueous composition. Alternatively, the liquid aqueous compositions may comprise from 0.5% by weight up to 20% by weight of the oil-soluble ascorbate to 100 parts by weight of total aqueous composition or liquid aqueous compositions may comprise from 1.0% by weight up to 18% by weight of the oil-soluble ascorbate to 100 parts by weight of total aqueous composition. In a further functional exemplary embodiment, an effective amount of ascorbic acid is an amount sufficient to reduce the number of N—Cl bonds in hair, on skin or in nails by at least about 50%, 60% or even 90% on a molecular number basis. I see that you are creating a broad spectrum of %'s

In further exemplary embodiments, a solution according to the disclosure may comprise a concentration of ascorbic acid ranging from about 0.5 to about 2 Molar. By way of non-limiting example, aqueous solutions may comprise about 0.3 to about 3 moles, such as about 0.5 to about 1.5 moles, or about 1.5 to about 3 moles, of compounds of formula A per one liter of water. Concentrates to be later mixed with water to compositions of these active levels of ascorbates may even have higher concentrations.

In exemplary embodiments where treatment compositions are applied to reduce or eliminate adverse effects of exposure to chlorinating and/or brominating agents, e.g., the odor of chlorine and/or itching and/or skin irritation, the effective amount of ascorbate may vary. Choosing an effective amount is within the skill of those of skill in the art, and may, for example, be done empirically, using more ascorbic acid where the odor of chlorine and/or itching/and/or skin irritation persists after applying an initial amount of ascorbate. By way of example only, an effective amount of ascorbate may range from about 0.2 to about 10 grams, such as about 0.5 to about 5 grams, or such as about 1 to about 3 grams per 0.1 liters of water. In certain of the various exemplary embodiments, the compositions and methods disclosed herein comprise ascorbate solutions having a pH of from 5.0-8.0 and preferable 5.5-7.8 and more preferably 6.0 to 7.5, although in treatment of fabric or surfaces, the pH may be less than about 6, such as, for example, less than about 5, less than about 4, or less than about 3. In further embodiments, the ascorbic acid solutions may have a pH of between about 3 and about 4. In yet further exemplary embodiments, the ascorbate solutions may have a pH of about 2. In one embodiment, the pH of the ascorbic acid solutions may range from about 1.9 to about 2.2. In further exemplary embodiments, the compositions and methods disclosed herein comprise ascorbate solutions, dispersions, suspensions or emulsions having a pH in the range of about 5 to about 8, such as about 5 to about 7.5, about 6 to about 7, or about 7 to about 7.5. Compositions having a particular pH and concentration of ascorbic acid may be made by applying the Henderson-Hasselbalch equation for estimating the pH of a buffer solution. Baby Shampoos, lotions and conditioners need to be pH balanced.

In yet further exemplary embodiments, the ascorbate used in the compositions and/or methods described herein may be replaced with, or used in combination with, one or more acid, salt, and/or derivatives thereof, to form acidic solutions, such as, for example, acidic aqueous solutions. Any acid, salt, and/or derivative thereof that is safe for the intended application may be used. For example, one or more of the following acids may be used in an acidic aqueous solution contemplated by the disclosure: acetic acid, citric acid, aconitic acid, adipic acid, benzoic acid, caprylic acid, cholic acid, desoxycholic acid, erythorbic acid, ferulic acid, formic acid, glutamic acid, glycocholic acid, hydrochloric acid, lactic acid, linoleic acid, malic acid, nicotinic acid, oleic acid, pectinic acid, phosphoric acid, propionic acid, sorbic acid, stearic acid, succinic acid, sulfamic acid, sulfuric acid, tannic acid, tartaric acid, taurocholic acid, and/or thiodipropionic acid. As a further example, one or more of the following salts may be used in the compositions according to the disclosure: lauryl sulfate salts, laureth sulfate salts, and sodium ascorbate. The compositions according to further embodiments may also comprise glycerin and/or menthol. As such, throughout the disclosure where compositions and methods comprising ascorbic acid, salts, and/or derivatives thereof are described, it should be understood that the ascorbate, salts, and/or derivatives may be replaced with, or used in combination with, one or more acid, salt, and/or derivative thereof.

Exemplary compositions according to the disclosure may be aqueous, and may be in any known form. For example, they may be solutions, powders (to later form aqueous compositions), tablets (to later form aqueous compositions), creams, gels, emulsions, etc. The compositions according to various embodiments of the disclosure may be packaged in any type of vessel or container that is useful for the specific formulation, and effective to dispense the composition. In at least certain exemplary embodiments, the vessel containing the composition according to the disclosure may be free or substantially free of O₂ for stability. A Bag on Valve, application. For example, the composition may be applied or administered by dispensing or spraying it from an aerosol or non-aerosol container that is free or substantially free of O₂.

In one embodiment outside the scope of the dispensers of the present invention, for example, the compositions may be in the form of a tablet, such as an effervescent tablet, which may be placed in water or another solvent before use. In yet a further exemplary embodiment, the composition may be an aqueous solution.

Exemplary soap compositions according to the disclosure may be made, for example, by mixing (A) an aqueous solution of ascorbate as described herein with (B) a liquid soap composition. In various embodiments, the soap compositions according to the disclosure have a pH of less than about 6, such as, for example, less than about 5, less than about 4, or less than about 3. In further exemplary embodiments, the soap compositions according to the disclosure have a pH in the range of about 5 to about 8, such as about 5 to about 7.5, about 6 to about 7, about 7 to about 7.5, or about 6. In another exemplary embodiment, the composition may be a foaming composition. Foaming compositions contemplated herein may comprise, inter alia (what is inter alia?), water, ascorbate, and a foaming agent.

Exemplary foaming compositions according to the disclosure may be made, for example, by mixing an aqueous solution of ascorbate with a foaming agent (e.g., an appropriate shampoo, soap, or body wash) to form a liquid that is capable of providing a foam when dispensed through a foaming hand soap dispenser. In various embodiments, the foaming compositions according to the disclosure have a pH of less than about 6, such as, for example, less than about 5, less than about 4, or less than about 3. In further exemplary embodiments, the foaming compositions according to the disclosure have a pH in the range of about 5 to about 8, such as about 5 to about 7.5, about 6 to about 7, about 7 to about 7.5, or about 6.

There are many competing forces and potentials within any aqueous solution comprising ascorbates, as in the present invention. Ascorbates are susceptible to oxidation at pHs below 5.7, and their solutions can oxidize at significant rates to produce undesirable levels of ascorbic acid. pH alone is not the issue, as the presence of an oxidizing agent in the solution is needed to produce this harmful effect. Therefore, the reduction of the concentration of oxygen to a level that is considered oxygen free (less than 0.5 mg/L H₂O at standard pressure (1 atmosphere) and temperature (20 °C) is desired. A level of less than 0.20 mg/L in the stable solution is even more desirable. It is even desirable to consider the amount of oxygen solely with respect to the ascorbate, with the total free oxygen within the aqueous deliverable composition to be maintained at a molecular ratio of less than 5% O₂/mol ascorbate, and preferably les than 1% O₂/mol ascorbate. The original solution placed into a dispenser may be slightly higher, as antioxidants in the solution, and some acceptable limits of oxidation (less than 2% of ascorbate oxidized) will occur after the ingredients are mixed, thus bringing the relative proportions of materials within an acceptable range, and this forming a stable solution that can be part of an emulsion in the dispenser.

The solubility of O₂ in water is temperature, pressure and pH dependent, such that at 0 °C (at on atmosphere of pressure) freshwater holds about 14.6 mg/L of oxygen, whereas at 25 °C, this drops to 8.3 mg/L.

The stability of ascorbates in water has been thoroughly discussed and described, as in Stability of aqueous solutions of ascorbate for basic research and for intravenous administration, Brett A. Wagner and Garry R. Buettner, Author manuscript; available in PMC: 2023 Dec. 1. Published in final edited form as: Adv Redox Res. 2023 Jul. 16; 9:100077.

The contained aqueous composition can be a solution or more likely an emulsion of a rapidly expanding liquid (upon release of ambient pressure, as during delivery) within another liquid, or less likely a high concentration of dissolved gas or liquid in the aqueous liquid. Where an emulsion is used, it is generally desirable (or even necessary for maximum use of components) to agitate the emulsion before delivery. This typically accomplished by vigorous shaking (by hand is sufficient) of the container with the emulsion components therein. This can typically be accomplished with 5-30 seconds of effort. Longer times may be used, but with no significant increase in efficiency of establishing a well distributed emulsion. The compositions of the present invention at pH levels of from 5.5-8.0 (preferably from 6.0-8.0 and more preferably from 6.6-7.8) can be dramatically more stable than standard compositions. In spite of the addition of potentially adversely impacting propellant by selecting not only a propellant based on critical properties impacting pH and oxidative capability (non-oxidizing propellant). The propellant itself should not be less than pH 6.0 (alone in distilled water), and should be neutral in its oxidizing capability. By using the higher pH propellants (greater than 6.5), the rate of oxidation of the ascorbate in a 5% oxygen environment is believed to be reduced by more than 25% as compared to the use of CO₂ (pH about 5.65) alone as a propellant. It is possible to blend propellants so that less expensive non-flammable propellants such as carbon dioxide can be used, as long as the propellant blend does not reduce the pH of water when dissolved therein as standard temperature and pressure. Propellant is used in any amount that allows for the desired liquid composition to be expelled as a gel, solution, suspension of foam. Typically the mixture has at least 5% propellant and less than 95% liquid composition. Preferably the total composition is 8-40% by weight propellant with the remainder the liquid composition (with some filler allowed). More preferably, the total composition is from 10-35% by weight propellant and up to 90% or up to 65% by weight of the dispenser-contained composition of liquid ingredient and propellant composition (with some filler allowed). Filler need not be included within the limits of the dispenser-contained or liquid composition with propellant, and functional/desirable components (e.g., antioxidants, skin softeners, fragrances, dyes, pigments, emulsifiers, suspension aids, and the like) may or may not be included within non-filler ingredients.

In addition to ascorbate, other component(s) useful in compositions according to the intended application, as long as the additional component(s) do not substantially interfere with the intended function of the ascorbic acid. By way of non-limiting example, for applications intended to be applied to body and/or clothing fibers, such additional components may include, but are not limited to, emollients, preservatives, perfumes, thickeners, cleansing agents, etc. It may also be desirable in various embodiments that the additional components do not damage or otherwise adversely affect the skin, hair and/or nails to which the composition is applied. Additional ingredients beneficial to skin, Vitamins A, D, B and E as well as Hyaluronic acid, moisturizing oils, essential oils can be added to the invention in solutions involving leave on scenarios. Because all Municipal water in showers and baths have chlorine as a disinfectant in water towers, use of the invention post shower/bath is necessary.

One of skill in the art would be able, through routine experimentation using the information provided herein, to formulate acceptable compositions comprising an effective amount of ascorbate, salts, and/or derivatives thereof, and formulations thereof, for treatment of chlorinated or chlorine-affected surfaces. As non-limiting examples, formulations comprising ascorbate in a molar ratio of about 79:1, or comprising water:ascorbate acid in a molar ratio of about 1:55.6.

Further aspects of the disclosure provide methods of mitigating adverse effects of exposure to oxidizers such as chlorinating and/or brominating agents, e.g. swimming pool chemicals and associated itching, irritation, and/or “pool odor.” The composition according to the disclosure may be sprayed by any method known, such as, for example, an aerosol spray, pour bottles, squeeze bottles or a non-aerosol pump bottle. In at least one exemplary embodiment, the composition according to the disclosure is applied by using a foaming composition comprising ascorbic acid, water, and a foaming agent. In some exemplary embodiments, the foaming composition is applied by dispensing said foaming composition through a dispenser (e.g. a foaming hand- or body-soap, or shampoo, dispenser), thereby creating a foaming lather, which is applied to the hair and/or skin. “No touch” dispensers can be used to administer a gel, liquid or foam of the invention.

In at least one exemplary embodiment of a subject exposed to chlorine and/or bromine in a swimming pool, the subject may optionally first rinse the residual pool water from his/her hair and/or body, then apply a composition according to the disclosure to his/her hair and/or body. In some embodiments, the person may thereafter immediately or substantially immediately wash the hair and/or body with soap, or may apply shampoo and/or conditioner. In further embodiments, the composition according to the disclosure is sprayed onto the hair and/or body after rinsing off residual pool water without subsequently immediately or substantially immediately washing the hair and/or body. This disclosure also contemplates applying a composition according to the disclosure without first rinsing. When a composition according to the disclosure is applied without first rinsing, larger quantities may, in at least some embodiments, be required than when applied subsequent to rinsing.

In at least one exemplary embodiment, the method of applying a composition according to the disclosure is intended to include a method where a subject, such as a swimmer, applies an amount sufficient to reduce and/or eliminate his/her own “pool odor” and/or irritation as perceived by the subject. For a particular aqueous solution according to the disclosure, the effective amount required to reduce and/or eliminate the subject's odor and/or irritation may depend on the amount of hair and/or skin that the subject has. Generally speaking, a subject may adjust the amount of the composition applied based on his or her own observations—if the subject experiences “pool odor” and/or irritation following administration, more may be applied. In various embodiments, the methods according to the disclosure relate to methods of treating hair comprising applying a composition as described herein. It is contemplated that a person with little or no hair would recognize that using a smaller amount of the composition provides effective reduction or elimination of “pool odor” and/or irritation, and thus, in at least certain embodiments, an effective amount may be less than in an embodiment where a person has a lot of hair and skin. (The use of dechlorination Glyceryl Ascorbates in post-shower, after bath, after pool/hottubs is necessary because 99% of all showers using municipal water contains chlorinated water.) After shower sprays, conditioners and lotions are helpful at lower levels. Likewise exposure to higher concentrations of halogens require stronger concentrations of the invention, double strength, triple strength etc.

The compositions according to the disclosure may, in various embodiments, be applied to the body and/or clothing fibers and immediately removed (e.g. by rinsing the body right away, that is in less than one minute), or may be left on the fibers for a period of time after application. For example, the body may be washed (e.g. with soap or shampoo) subsequent to the application of the composition.

As described herein, the disclosure is set forth with regard to “people” for ease of reference only. However, the invention is not limited to humans, but rather is intended to include any mammal having hair. As such, the use of the terms “swimmer” or “people” is intended to include any mammal that swims or is otherwise exposed to swimming pool chemicals such as chlorine and/or bromine.

The terms “pool chemicals” and the like are used generally throughout the disclosure for ease of reference, but should not be considered limited to chemical exposure in a swimming pool. Rather, any chemical such as chlorinating agents (e.g., hypochlorite salts such as calcium hypochlorite or sodium hypochlorite, hypochlorous acid) or brominating agents (e.g., bromine, salts comprising bromine, hypobromous acid, etc.) or other oxidative halogens are intended to be included with reference to “pool chemicals” and the like, whether or not a person is actually exposed to those chemicals in a swimming pool.

The term “biological fibers” is intended to refer to fibers comprising proteins, such as those found in and from living beings, including but not limited to skin, hair, nails, muscle, and other fibrous tissues of mammals, as well as plant fibers. It is also intended that fibers from harvested animals that are no longer living, such as harvested poultry during processing, are included in the term “biological fibers,” as these fibers likewise comprise proteins.

Further embodiments of the disclosure are related to methods for treating biological and/or synthetic fibers, such as may be found in some textiles, e.g. towels, swimming attire, swimming accessories, etc., exposed to oxidizing agents, such as those exposed to chlorinated and/or brominated pool water, with an aqueous solution of ascorbic acid. Such treatment may reduce the “pool odor” of, and/or “wear” associated with such exposure on, the textiles. For example, treating swimming attire exposed to chlorinated and/or brominated pool water with a composition according to the disclosure has been found to reduce the “pool odor” of that swimming attire. Treating swimming attire exposed to chlorinated and/or brominated pool water with composition according to the disclosure has also been found to reduce the oxidative damage done to the swimming attire, thereby prolonging its life and reducing the fading and/or discoloration of its materials, typically associated with such textiles. As used herein, the terms “clothing” and “textiles” should be considered interchangeable in that when the term “clothing” is used, it is intended to include any textiles within the scope of the disclosure. For example, when reference is made to treatment of a swimmer's clothing or attire, it is intended that a swimmer's towel, etc., is included in said disclosure and claims. It should also be noted that textiles are intended to be included as biological and/or synthetic fibers, as textiles are traditionally made up of fibers such as plant and/or animal fibers, or may be manmade, such as polymer fibers.

In at least one exemplary method, an effective amount of a composition according to the disclosure is applied by treating the textiles with an aqueous solution of ascorbate, such as by applying the composition by contacting the textiles with said composition. Any method for treating textiles known in the art may be used, such as, for example, spraying the textile with the composition using an aerosol or non-aerosol spray; immersing the textile into the composition; and/or washing the textile with a soap composition comprising ascorbic acid, as described herein. In one exemplary embodiment, the textiles may optionally first be rinsed with standard tap water before applying the composition according to the disclosure, and optionally thereafter be rinsed a second time with standard tap water after the application. In another embodiment, the composition according to the disclosure is applied without first rinsing the textiles with standard tap water. The textiles may optionally be washed as normal, subsequent to the application of the composition according to the disclosure. In at least one exemplary embodiment, the textiles may be treated with about 0.2 to about 10 grams of ascorbate carried in water, such as about 0.5 to about 5 grams of ascorbate, or about 1 to about 3 grams of ascorbate.

In further embodiments according to the disclosure, methods for reducing biological fibers are also contemplated. Such methods include steps of (1) identifying biological fibers which are capable of undergoing reduction, and (2) applying a composition according to the disclosure to said biological fibers. In further embodiments, said methods include a step of applying a composition according to the disclosure to said biological fibers.

In further embodiments according to the disclosure, methods of administering vitamin C to a mammal are contemplated, said method comprising administering a composition according to the disclosure via a vessel that is free or substantially free of O₂. Ascorbic acid is known to degrade when allowed to stand in the presence of oxygen. Oxygen is a natural component of the Earth's atmosphere, where it resides in air and water. It has been found that storing the compositions according to the disclosure in an oxygen-free environment provides for many advantages, such as prolonging storage and shelf life.

Aqueous ascorbate gels, emulsions or solutions according to various embodiments of the disclosure may thus optionally be made oxygen-free, for example by bubbling an inert gas through the solution, thereby driving out any oxygen dissolved in the water. Alternatively, aqueous ascorbate solutions may be made by mixing the ascorbate with water, sealing the solution off from the environment (e.g., by sealing the solution in a bottle), and allowing the bottle to stand until the oxygen sealed inside the bottle is consumed by the ascorbate in the solution. By way of example only, methods of administering ascorbate to mammals may comprise dispensing a composition according to the disclosure by spraying said composition, e.g. using an aerosol spray or a non-aerosol pump, foaming chamber, (Aloe lotions with glyceryl Ascorbate(s)), Shaving creme, Suntan lotion, and After-Sun lotions.

Maintaining free chlorine in recommended levels is the most important activity in terms of ensuring your pool and hot tub are clean and safe to use. However, recommended levels are not necessarily the same for both pools and hot hubs. Further, recommended levels are dependent on cyanuric acid, temperature and bather load. And the effectiveness of free chlorine's ability to kill contaminants is greatly dictated by the pH level of the water.

### Ideal Levels of Free Chlorine, Combined Chlorine, and Total Chlorine

The Association of Pool and Spa Professionals recommends free chlorine levels be kept between 2.0 and 4.0 ppm. The Center for Disease Control recommends free chlorine stay above 1 ppm in pools and 3 ppm in hot tubs. Municipal water towers are required to keep chlorine above 0.5 ppm to guard against Legionella and other pathogens, most cities maintain between 1 and 2 ppm.

The easiest way to check chlorine levels is with test strips. Free chlorine is the amount of chlorine freely available to kill bacteria and other contaminates. Chlorine bound by contaminates are called Combined Chlorines, some of these can be very toxic.

The recommended amount of “combined chlorine” is no more than 0.2 ppm. Combined chlorine or chloramines is the portion of chlorine that has reacted with the water and microorganisms contained in it. It is a temporary form of chlorine that should be oxidized if there is at least than 7.5 ppm of free chlorine in the water to burn up the contaminates. Too much combined chlorine (i.e. more than 0.5 ppm) means that there is not enough free chlorine available to kill all the contaminates and causes severe skin, lung and eye irritation as it is emitted from the pool. When combined chlorine reaches these levels, the water may cause skin and eye irritation, be harmful to swim in due to bacteria, and may allow algae to form. The present technology in sufficient concentration will also attack and destroy the more complex N—CL bonds of Combined Chlorine.

The present technology has a rapid and significant effect on the chlorine products in hair (which is the easiest to determine standards because of greater absorption consistency), and these tests may also be used with skin products. For example, human hair has an average thickness of from 17 to 181 micrometers (millionths of a meter). A basic test would be for human hair having an average thickness of from 50-100 micrometers to be immersed in chlorinated water having 2 ppm free chlorine and less than 0.5 ppm combined chlorine for 15 minutes. This hair (whether removed from a human scalp and retained in a clip at one end or still on the head of a human) is then treated (washed or soaked) for 1.5-2.0 minutes with the hair (or skin) treating composition of the invention and rinsed thoroughly in tap water with scrubbing to remove at least 95% by weight of the applied composition. The hair is then leached in 1.0 liters of deionized water for 2 minutes and the free chlorine level and the combined chlorine level measured. The free chlorine level must be below 0.1 ppm and the combined chlorine level must be below 0.05 ppm to pass this test. An additional test can be to administer the present technology to a solution of chlorinated/brominated water, with concentration of greater 2 ppm and less than 0.5 ppm of combined chlorine. Effectiveness can be measured by the time required for the anti-oxidation of the halogens in the water. This test can be compared and contrasted to the extant products on the market.

Although the present disclosure herein has been described with reference to various exemplary embodiments, it is to be understood that these embodiments are merely illustrative of the principles and applications of the present disclosure. Those having skill in the art would recognize that various modifications to the exemplary embodiments may be made, without departing from the scope of the invention. Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

It will be appreciated that there is an implied “about” prior to all numerical values recited herein, whether or not so stated. It should also be understood that the precise numerical values used in the specification and claims form additional embodiments. Efforts have been made to ensure the accuracy of the numerical values disclosed herein. Any measured numerical value, however, can inherently contain certain errors resulting from the standard deviation found in its associated measuring technique. As used herein, reference to percent is intended to indicate wt %, relative to the weight of the composition.

Furthermore, other embodiments of the disclosure will be apparent to those skilled in the art from consideration of the specification and practice of the invention disclosed herein. It is intended that the specification and examples be considered as exemplary only, with a scope and spirit being indicated by the claims.

Finally, it is noted that, as used in this specification and the appended claims, the singular forms “a,” “an,” and “the,” include plural referents unless expressly and unequivocally limited to one referent, and vice versa. Thus, by way of example only, reference to “a composition” can refer to one or more compositions, and reference to “a salt of ascorbate” can refer to one or more salts of ascorbate. As used herein, the terms “comprise”, “comprises”, “comprising”, “contain”, “contains”, “containing”, “have”, “having”, “include”, “includes”, and “including” are intended to be non-limiting, such that recitation of an item or items is not to the exclusion of other like items that can be substituted or added to the recited item(s).

### EXAMPLES

The following examples are illustrative only, and are not intended to be limiting of the invention, as claimed.

### Example 1

At standard temperature and pressure, 330 grams of bisglyceryl ascorbate or its sodium salt were dissolved in 1 L of water. This solution 1 was then applied to human hair and human skin that had been exposed to swimming pool chemicals, oxidizers or halogens.

### Example 2

At standard temperature and pressure, 300 grams of bisglyceryl ascorbate were dissolved in 1 L of water along with coco betaine. This solution 2 was then applied to hair and human skin that had been exposed to swimming pool chemicals.

### Example 3

At standard temperature and pressure, 270 grams of ascorbate were dissolved in 1 L of water along with citric acid. This solution 3 was then applied to human skin and human hair that had been exposed to swimming pool chemicals.

### Example 4

At standard temperature and pressure, 240 grams of ascorbic acid were dissolved in 1 L of water along with an emulsifying agent and lemon oil. This solution 4 stored in a hand spray pump bottle for three days and then was then applied to human skin and human hair that had been exposed to swimming pool chemicals.

### Example 5

At standard temperature and pressure, 180 grams of ascorbic acid were dissolved in 1 L of water. This solution 6 was then applied to human hair and human skin that had been exposed to swimming pool chemicals.

### Example 6

At standard temperature and pressure, 180 grams of ascorbic acid were dissolved in 1 L of water along with an emulsifying agent, coco betaine and peppermint oil. The resulting solution was transferred into 3 oz spray bottles. Following swimming in a chlorinated swimming pool, a subject's skin and hair were rinsed with warm shower water for about 30 seconds. Then, the solution 6 was applied to the subject by spraying the solution onto the skin and hair. A total of 10 ml of solution 6 was evenly sprayed onto the subject's skin, and a total of 20 mL of the solution was evenly sprayed onto the subject's hair. The subject was then allowed to wash and rinse their skin with soap, and shampoo, rinse, and condition their hair, as normal.

The shampoo was applied to the hair without first rinsing out the solution 6, so that the shampoo and lather distributed the solution.

Other ingredients may include propylene glycol, sodium benzoate, glycerine, DMDM (3′-Demethoxy-o-Demethylmatairesinol), hydantoin imadazolininyl, sodium benzoiate, 2-bromo-2-nitropropane-1-2-diol. There are a whole range of products dechlorinating in the line of compositions useful in the disclosed invention, such as in shampoo, Hand Soap, Body-Wash, Bubble bath, Baby Shampoo (liquid, gel, foam); shampoo and conditioner; Conditioner, Leave in Conditioner, De-tangler, after shower spritz (liquid conditioner sprays); Dechlorinating Lotion moisturizer, Aloe gel, shaving creme, shaving foam, wet-wipes and the like. Additions to the formulae include many special materials that are good for skin such as Vitamin A & D, anti-oxidant Vitamins C and E, as well as Hyaluronic acid, essential oils etc.

Optionally, there may be the addition of Vitamin A, D, B, E, hyaluronic acid, essential oils? Both in Soap and Conditioner and lotion forms. A maximum effective formula would use a wide range of materials such as the basic formulations of the above examples with using anything else that chemically address chlorine activity or even the chlorine/Bromine bonds, such as thiosulfate salts, chelating agents for chlorine and other ascorbates.

Propellant-based aerosol delivery of foamed compositions of the invention requires combined expertise in many different disciplines. The use of foam delivery allows for the application of ‘controllable’ foam to a part of the body that the user might not be able to visually see during application. Where thick creams could coat the hair before reaching the scalp and serums might prove too thin to be able to easily apply and control, a light foam helps to bridge the gap between ease of application and penetration to the scalp. Increasingly, we can expect foams to become the dominant product form for many different over the counter and Rx scalp treatments.

Foams intended to be applied to the skin can also provide an increased level of manual control and lightness of application. A great example of a well-designed topical foam can be found in aerosol packages with menthol delivering a moderately stiff foam that results in easy and quick application, quickly collapsing and allowing for a thin, fast-drying layer without the sensation of heaviness or greasiness. The result is a pleasant and highly effective topical pain-relieving experience.

Over the past few years, an even more unique dispensing approach can be found in the eN₂fused™ Whipped Dispensing Technology offered by Formulated Solutions. This patented technology gives Formulated Solutions the ability to create a topical lotion, cream, or ointment and then saturate the formula with compressed gas, with the gas molecules occupying all the interstitial spaces between all the formula molecules. The company then infuses a highly stable compressed gas microbubble structure into the formula and holds it under pressure within a bag-on-valve pressurized container system. When dispensed, the result is a lotion, cream or ointment that transforms into a whipped cream structure, only it needn't be dispensed upside down.

Each of these foam aerosol drug dispensing formats helps to deliver a more ideal consumer experience through either increasing product ‘control’, enhancing spreadability, or creating a more luxurious or gentle application. By including aerosol foam and whipped applications in the formulator's toolbox, we can effectively increase the ways we deliver on both drug delivery convenience and compliance within the directed consumer self-care category.

An aerosol formulation typically consists of two components: the product concentrate and the propellant. The product concentrate is the active drug or chemistry combined with additional ingredients or co-solvents required to make a stable and efficacious product. This may also include foaming agents. The concentrate can be a solution, suspension, emulsion, gel, or (not within the scope of the present invention) a semisolid, or powder. The propellant provides the force that expels the product concentrate from the container and additionally is responsible for the delivery of the formulation in the proper form (i.e., spray, foam, semisolid). When the propellant is a liquefied gas or a mixture of liquefied gases, it can also serve as the solvent or vehicle for the product concentrate. If the product characteristics are to change on dispensing, additional energy in the form of a mechanical breakup system may be required.

### Propellants

A propellant is a chemical with a vapor pressure greater than atmospheric pressure at 40 °C (105 °F). Types of propellants commonly used in pharmaceutical aerosols include chlorofluorocarbons, hydrocarbons, hydrochlorofluorocarbons and hydrofluorocarbons, and compressed gases such as NO₂, N₂, CO₂ and the like.

### Chlorofluorocarbon (CFC) Propellants

For many years, the chlorofluorocarbon (CFC) propellants P-11, P-12, and P-114 were used in aerosol products. Their use has been severely curtailed due to their role in depleting the ozone layer of the atmosphere. Since January 1996, worldwide production of these CFCs has been reduced to only the amount needed for aerosols used in the treatment of asthma and chronic obstructive pulmonary disease. Alternatives to P-12 (i.e., P-134a and P-227) have now been developed and are being incorporated in aerosol formulations. Currently, there are not alternatives for P-11 and P-114. Small amounts of P-11 are required in most aerosol suspensions to make a slurry of the active drug and other ingredients. It also is used to dissolve surfactants in some formulations.

P-11, P-12, and P-114 are the CFCs of choice for oral, nasal, and inhalation aerosols. These particular chlorofluorocarbon propellants are well accepted due to their relatively low toxicity and inflammability. The chlorofluorocarbons as a class are inert but P-11 is subject to hydrolysis and will form hydrochloric acid in the presence of water. The acid increases the corrosion of the container and may be irritating when applied to membranes. If water is present, P-12 or a mixture of P-12 and P-114 are used. It may also interact with ingredients in the concentrate, deteriorating intended performance.

The CFCs are gases at room temperature that can be liquefied by cooling them below their boiling point or by compressing them at room temperature. For example, dichlorodifluoromethane (P-12) will form a liquid when cooled to −21.6 °F or when compressed to 84.9 psia at 70 °F (psia=pounds per square inch absolute). These liquefied gases also have a very large expansion ratio compared to the compressed gases (e.g., nitrogen, carbon dioxide). The usual expansion ratio for liquefied gases is about 1 to 240 which means that 1 mL of liquefied gas will occupy a volume of approximately 240 mL if allowed to vaporize. Compressed gases have an expansion ratio of about 3 to 10.

| | | | | Liquid |
|---|---|---|---|---|
| | | V.P. | | Density |
| | | @70 °F | B.P. | @70 °F |
| Name | Formula | (psia)^{a} | °F (1 atm) | (g/mL) |
| Trichloromono- | CCl₃F | 11 | 13.4 | 74.7 |
| fluoromethane | | | | |
| Dichlorodi- | CCl₂F₂ | 12 | 84.9 | −21.6 |
| fluoromethane | | | | |
| Dichlorotetra- | CClF₂ClF₂ | 114 | 27.6 | 39.4 |
| fluoroethane | | | | |

*psia (pounds per square inch absolute) = psig (pounds per square inch gauge + 14.7)

When a liquefied gas propellant or propellant mixture is sealed in an aerosol container with the product concentrate, an equilibrium is establish between the propellant which remains liquefied and a portion that vaporizes and occupies the upper portion of the container. The pressure at this equilibrium is referred to as the vapor pressure (expressed as psia) and is a characteristic of each propellant at a given temperature. Since the vapor pressure is exerted equally in all directions and is independent of the quantity of liquefied phase present, the pressure forces the liquid phase up the dip tube and out of the container when the valve is actuated. As the propellant reaches the air, it evaporates due to the drop in pressure and leaves the product concentrate as airborne liquid droplets or dry particles or may be temporarily retained as the gas within a foamed delivery. As the liquid is removed from the container through the dip tube, the equilibrium between the propellant's liquefied phase and vapor phase is rapidly re-established. Thus, the pressure within the container remains virtually constant and the product may be continuously released at an even rate and with the same propulsion.

In the case when there is no dip tube in the container, the container is used in the inverted position so that the liquid phase will be in direct contact with the valve. When the valve is actuated, the liquid phase is emitted and immediately reverts to the vapor phase in the atmosphere.

### Hydrochlorofluorocarbons (HCFC) and Hydrofluorocarbons (HFC)

The hydrochlorofluorocarbons (HCFC) and hydrofluorocarbons (HFC) differ from CFCs in that they may not contain chlorine and have one or more hydrogen atoms. These compounds break down in the atmosphere at a faster rate than the CFCs resulting in a lower ozone depleting effect.

### Hydrocarbons

The hydrocarbons are used in topical pharmaceutical aerosols because of their environmental acceptance and their low toxicity and nonreactivity. They are also useful in making three phase (two layer) aerosols because of their density being less than 1 and their immiscibility with water. The hydrocarbons remain on top of the aqueous layer and provide the force to push the contents out of the container. However, they are flammable and can explode. They contain no halogens and therefore hydrolysis does not occur making these good propellants for water based aerosols. These hydrocarbon propellants typically include propane, isobutane and butane.

Propane, butane, and isobutane are the most commonly used hydrocarbons. They are used alone or as mixtures or mixed with other liquefied gases to obtain the desired vapor pressure, density, and degree of flammability. The flammability hazard has been substantially reduced by using mixtures of propellants and with the development of newer types of dispensing valves (i.e., valve with vapor tap).

### Compressed Gases

Gases such as nitrogen, nitrous oxide, and carbon dioxide have been used as aerosol propellants for products dispensed as fine mists, foams, or semisolids. But due to their low expansion ratio, the sprays are fairly wet and the foams are not as stable as produced by liquefied gas propellants. However, using a compressed gas that is insoluble in the product concentrate (e.g., nitrogen) will emit the product concentrate in essentially the same form as it was placed in the container.

The pressure of the compressed gas contained in the headspace of the aerosol container forces the product concentrate out of the container. But unlike aerosols prepared with liquefied gas propellants, there is no propellant reservoir. So higher gas pressures are required in these aerosols and the pressure diminishes as the product is used. These gases have been used for the most part to dispense food products, dental creams, hair preparations, and ointments.

### Product Concentrates

Solution aerosols are two phase systems consisting of the product concentrate in a propellant, a mixture of propellants, or a mixture of propellant and solvent. Solvents may also be added to the formulation to retard the evaporation of the propellant. Solution aerosols can be difficult to formulate because many propellant or propellant-solvent mixtures are nonpolar and are poor solvents for the product concentrate. Also, there is a limited number of solvents that can be used. Ethyl alcohol is the most commonly used solvent but propylene glycol, dipropylene glycol, ethyl acetate, hexylene glycol, and acetone have also been used.

Aerosol solutions have been used to make foot preparations, local anesthetics, spray-on protective films, anti-inflammatory preparations, and aerosols for oral and nasal applications. They contain 50 to 90% propellant for topical aerosols and up to 99.5% propellant for oral and nasal aerosols. As the percentage of propellant increases, so does the degree of dispersion and the finest of the spray. As the percentage of propellant decreases, the wetness of the spray will increase. The particle sizes of the sprays can vary from 5 to 10 μm in inhalation aerosols and 50 to 100 μm for topical sprays.

Suspensions aerosols can be made when the product concentrate is insoluble in the propellant or mixture of propellant and solvent, or when a co-solvent is not desirable. Anti-asthmatic drugs, steroids, and antibiotics are delivered as suspension aerosols. When the valve is actuated, the suspension formulation is emitted as an aerosol and the propellant rapidly vaporizes and leaves a fine dispersion of the product concentrate.

Formulation considerations for suspension aerosols, not necessary with solution aerosols, include agglomeration, particle size growth, valve clogging, moisture content, and particle size of the dispersed aerosolized particles. Lubricants such as isopropyl myristate and light mineral oil, and surfactants such as sorbitan trioleate, oleic acid, and lecithin have been used to overcome the difficulties of particle size agglomeration and growth which are directly related to the clogging problems. The moisture content of the entire formulation should be kept below 200 to 300 ppm so all of the ingredients need to be the anhydrous form of the chemical or be capable of becoming anhydrous after a drying process. The particle size of the insoluble product concentrate ingredients should be in the 1 to 10 μm range for inhalation aerosols and between 40 to 50 μm for topical aerosols.

The product concentrate in an emulsion aerosol will consist of the active ingredient, aqueous and/or nonaqueous vehicles, and a surfactant. Depending on the components, the emitted product can be a stable foam (shaving cream type) or a quick breaking foam. A quick breaking foam creates a foam when emitted from the container but the foam collapses in a relatively short time. This type of foam is used to apply the product concentrate to a large area without having to manually rub or spread the product. Also, the active drug is more rapidly available because the foam quickly collapses.

Foams are produced when the product concentrate is dispersed throughout the propellant and the propellant is in the internal phase; i.e., the emulsion behaves like o/w emulsions. When the propellant is in the external phase (i.e., like a w/o emulsion), foams are not created but sprays or wet streams result. Stable foams are produced when surfactants are used that have limited solubility in both the organic and aqueous phases. Surfactants concentrate at the interface between the propellant and the aqueous phase forming a thin film referred to as the “lamella.” It is the specific composition of this lamella that dictates the structural strength and general characteristics of the foam. Thick and tightly layered lamellae produce very structured foams which are capable of supporting their own weight.

Surfactants used in emulsion aerosols have included fatty acids saponified with triethanolamine, anionic surfactants, and more recently nonionic surfactants such as the polyoxyethylene fatty esters, polyoxyethylene sorbitan esters, alkyl phenoxy ethanols, and alkanolamides. The nonionic surfactants are present fewer compatibility problems because they charge no electronic charge.

### The Valve Assembly

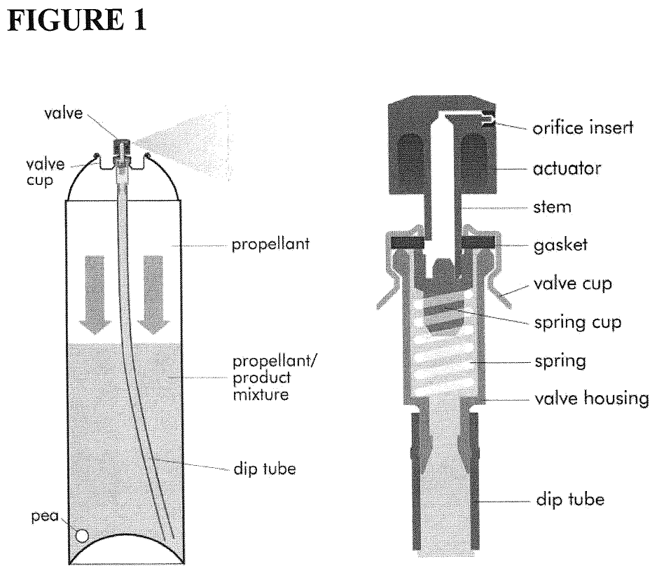

It is helpful to refer to FIG. 1 in considering this disclosure. The effectiveness of a pharmaceutical aerosol depends on achieving the proper combination of product concentrate formulation, container, and valve assembly. The valve mechanism is the part of the product package through which the contents of the container are emitted. The valve must withstand the pressure required by the product concentrate and the container, be corrosive resistant, and must contribute to the form of the emitted product concentrate.

The primary purpose of the valve is to regulate the flow of product concentrate from the container. But the valve must also be multifunctional and regulate the amount of emitted material (metered valves), be capable of delivering the product concentrate in the desired form, and be easy to turn on and off. Among the materials used in the manufacture of the various valve parts are plastic, rubber, aluminum, and stainless steel.

The basic parts of a valve assembly can be described as:
1. Actuator—the actuator is the button which the user presses to activate the valve assembly and provides an easy mechanism of turning the valve on and off. In some actuators, mechanical breakup devices are also included. Itis the combination of the type and quantity of propellant used and the actuator design and dimensions that determine the physical form of the emitted product concentrate.
2. Stem—the stem supports the actuator and delivers the formulation in the proper form to the chamber of the actuator.
3. Gasket—the gasket, placed snugly with the stem, serves to prevent leakage of the formulation of the valve is in the closed position.
4. Spring—the spring holds the gasket in place and is the mechanism by which the actuator retracts when pressure is released thereby returning the valve to the closed position.
5. Mounting Cup—the mounting cup which is attached to the aerosol container serves to hold the valve in place. Because the undersigned of the mounting cup is exposed to the formulation, it must receive the same consideration as the inner part of the container with respect to meeting criteria of compatibility. If necessary, it may be coated with an inert material to prevent an undesired interaction.
6. Housing—the housing located directly below the mounting cup serves as the link between the dip tube and the stem and actuator. With the stem, its orifice helps to determine the delivery rate and the form in which the product is emitted.
7. Dip Tube—the dip tube which extends from the housing down into the product concentrate serves to bring the formulation from the container to the valve. The viscosity of the product and its intended delivery to rate dictate the inner dimensions of the dip tube and housing for a particular product.

Spray valves are used to obtain fine to coarse wet sprays. Depending on the formulation and the design of the valve and actuator, the particle size of the emitted spray can be varied. The spray is produced as an aerosol solution passes through a series of small orifices which open into chambers that allow the product concentrate to expand into the proper particle size.

Vapor tap valves are used with powder aerosols, water based aerosols, aerosols containing suspended materials, and other agents that would tend to clog a standard valve. This valve is basically a standard valve except that a small hole has been placed into the valve housing. This allows vaporized propellant to be emitted along with the product concentrate and produces a spray with greater dispersion. These valves are used with aqueous and hydroalcoholic product concentrates and hydrocarbon propellants.

Foam valves have only one orifice that leads to a single expansion chamber. The expansion chamber also serves as the delivery nozzle or applicator. The chamber is the appropriate volume to allow the product concentrate to expand into a ball of foam. Foam valves are used for viscous product concentrates such as creams and ointments because of the large orifice and chamber. Foam valves also are used to dispense rectal and vaginal foams. If the size of the orifice and expansion chamber are appropriately reduced, a product concentrate that would produce a foam will be emitted as a solid stream. In this case, the ball of foam begins to develop where the stream impinges on a surface.

Metered dose inhaler (MDI) valves (metering valves) are used to accurately deliver a dose of medication. Metered valves are used for all oral, inhalation, and nasal aerosols. The metered valves reproducibly deliver an amount of product concentrate accurately from the same package and also allow for the same accuracy between different packages.

The amount of material emitted is regulated by an auxiliary valve chamber of fixed capacity and dimensions. This metering chamber volume can be varied so that about 25 to 150 mL of product concentrate is delivered per actuation. Access in and out of the metering chamber is controlled by a dual valve mechanism. When the actuator is closed, a seal blocks emission from the chamber to the atmosphere. However, the chamber is open to the contents of the container and it is filled. When the actuator is depressed, the seals reverse function; the chamber becomes open to the atmosphere and releases its contents and at the same time becomes sealed from the contents of the container. When the actuator is again closed, the system prepares for the next dose.

Two basic types of metering valves are available; one for inverted use and the other for upright use. Generally the valves for upright use are used with solution type aerosols and contain a thin capillary dip tube. Suspension or dispersion aerosols use the valve intended for inverted use that does not contain a dip tube.

In general, valves should retain the material in the metering chamber for fairly long periods. However, it is possible for the material in the chamber to slowly return back to the container. The degree to which this occurs depends on the construction of the valve and length of time between actuations of the valves. Some valves have been fitted with a “drain tank” to overcome this problem.

### Containers

Aerosol containers are generally made of glass, metals (e.g., tin plated steel, aluminum, and stainless steel), and plastics. The selection of the container for a particular aerosol product is based on its adaptability to production methods, compatibility with the formulation, ability to sustain the pressure necessary for the product, the design and aesthetic appeal, and the cost.

When liquefied gases (CFC, HCFC, HFC, hydrocarbons) are used as propellants, one of two systems can be formulated. The two phase system is the simplest system. Here the product concentrate is dissolved or dispersed in liquefied propellant and solvents creating a homogenous system. The propellants exist in both the liquefied phase and the vapor phase. When the aerosol valve is actuated, some liquefied propellant and solvent containing the product concentrate is emitted from the container. These aerosols are designed to produce a fine mist or wet spray by taking advantage of the large expansion of the propellant when it enters room temperature and atmospheric pressure. The two phase system is commonly used to formulate aerosols for inhalation or nasal application.

A three phase system (i.e., a heterogeneous system) is made up a layer of water immiscible liquid propellant, a layer of propellant immiscible liquid (usually water) which contains the product concentrate, and the vapor phase. This type of system is used when the formulation requires the presence of a liquid phase that is not propellant miscible. When the aerosol valve is actuated, the pressure of the vapor phase causes the liquid phase to rise in the dip tube and be expelled from the container. If the product is to maintain the liquefied gas reservoir, the dip tube must not extend beyond the aqueous phase. Sometimes it is desirable to have some liquefied propellant mixed with the aqueous phase to facilitate in the dispersion of the spray or to create a foam. In this case, the container should be shaken immediately prior to use.

If CFCs, HCFCs, and HFCs are used as the propellants, they will reside on the bottom of the container since their density is greater than water. The dip tube will then need to end somewhere in the middle of the container. If hydrocarbons are used as the propellants, they will reside on the aqueous layer since their density is less than water. In this case, the dip tube can be extended through the liquid propellant all the way down to the bottom of the container. Thus an important characteristic of any aerosol is the density of the propellant, propellants, or blend of propellants.

Foam aerosols are a three phase system in which the liquid propellant is emulsified with the product concentrate. When the valve is actuated, the emulsion is forced through the nozzle and the entrapped propellant reverts to the vapor phase and whips the emulsion into a foam when it reaches the atmosphere. To facilitate the formulation of a foam, some aerosols are shaken prior to use to disperse some of the propellant throughout the product concentrate. If a dip tube is present, the container is used while being held upright. If there is no dip tube, the container must be inverted prior to use.

Foam products operate at a pressure of about 40 to 50 psig at 70 °F and contain about 4 to 7% propellant. Generally, a blend of propane and isobutane is used for foam aerosols. Contraceptive foam aerosols use A-31 as the propellant. Other foams use P-152a since it will produce a more stable foam and is less flammable than hydrocarbons. Other propellants that have been used include the compressed gases nitrous oxide and carbon dioxide. Typical products include whipped creams and toppings and several pharmaceutical and veterinary products.

Aerosols using compressed gases as the propellant operate essentially as a pressure package. The pressure of the gas forces the product concentrate out of the container in essentially the same form as it was placed in the container. Only the product concentrate is expelled; the compressed gas remains in the container occupying the headspace. The pressure drops in the container as the product concentrate is removed and the gas expands to occupy the newly vacated space. The pressure will continue to drop as the product concentrate is expelled. Therefore, the initial pressure in these containers is higher than used in liquefied gas aerosols and is usually 90 to 100 psig at 70 °F. The amount of product left in the container after the pressure is exhausted varies with the viscosity of the product and loss of pressure due to gas seepage.

Depending on the nature of the formulation and the type of compressed gas used, the product may be dispensed as a semisolid (solid stream) foam or spray. Semisolid aerosols are used to dispense more viscous concentrates such as dental creams, hair dressings, ointments, creams, cosmetic creams, and foods.

In barrier pack systems, the propellant is physically separated from the product concentrate. The propellant pressure on the outside of the barrier serves only to push the contents from the container. In the piston type system, a polyethylene piston is fitted into the container. The product concentrate is placed into the upper portion of the container and a compressed gas or hydrocarbon gas is placed on the other side of the piston. The gas pushes against the piston and pushes the product concentrate out of the container when the valve is actuated. As the rises in the container, it scrapes against the side of the container which helps dispense most of the product concentrate.

This system is used to dispense cheese spreads, cake decorating icings, and ointments. Since these product concentrates are semisolid and viscous, they emit from the container as a lazy stream rather than a foam or spray. The piston type system is limited to viscous materials since liquids tend to pass around the edges of the piston into the gas compartment.

A collapsible plastic bag fitted into a container is another type of barrier pack system, but is not used in the present invention. In some systems, the bag is a thin walled aluminum pouch. The product concentrate is placed in the bag and the propellant surrounds the bag. The bag is accordion pleaded to prevent the gas from pinching it closed. These types of systems are used to dispense liquids as fine mists or streams, and semisolids as streams. They system can also be used for topical creams, ointments, or gels.

Gels that foam after being dispensed are placed in both the piston type and collapsible plastic bag type of system. The dispensed gel contains a low boiling liquid such as isopentane or pentane. The liquid will vaporized when the gel is placed in the warmth of the hands, producing the foaming gel.

Aerosols are unique among the pharmaceutical dosage forms because they depend on the function of a container, its valve assembly, and propellants for the physical delivery of the ingredients. The aerosol container is referred to as a pressurized package. The pressure inside the package is created by the presence of one or more liquefied or gaseous propellants. When the valve is actuated, the pressure forces the contents of the package out through the opening in the valve. The physical form of the expelled contents is a function of the product formulation and the type of valve employed.

Aerosols used to provide an airborne mist are called space sprays and include room disinfectants and deodorizers. This group of aerosols produce particles that are usually less than 50 μm in size. This will ensure that the dispersed droplets or particles will remain airborne for a prolonged period of time. A one-second burst from a typical space spray will produce 120 million particles, of which a substantial number will remain airborne for an hour.

Aerosols intended to carry the active drug to a surface are called surface sprays or surface coating sprays. This class of sprays includes products such as deodorant sprays, hair sprays, perfume and cologne sprays, shaving lathers, paint sprays, and various household products such as spray starch, waxes, polishes, and cleaners.

### Product Concentrates

Solution aerosols are two phase systems consisting of the product concentrate in a propellant, a mixture of propellants, or a mixture of propellant and solvent. Solvents may also be added to the formulation to retard the evaporation of the propellant. Solution aerosols can be difficult to formulate because many propellant or propellant-solvent mixtures are nonpolar and are poor solvents for the product concentrate. Also, there is a limited number of solvents that can be used. Ethyl alcohol is the most commonly used solvent but propylene glycol, dipropylene glycol, ethyl acetate, hexylene glycol, and acetone have also been used.

Aerosol solutions have been used to make foot preparations, local anesthetics, spray-on protective films, anti-inflammatory preparations, and aerosols for oral and nasal applications. They contain 50 to 90% propellant for topical aerosols and up to 99.5% propellant for oral and nasal aerosols. As the percentage of propellant increases, so does the degree of dispersion and the finest of the spray. As the percentage of propellant decreases, the wetness of the spray will increase. The particle sizes of the sprays can vary from 5 to 10 μm in inhalation aerosols and 50 to 100 μm for topical sprays.

Suspensions aerosols can be made when the product concentrate is insoluble in the propellant or mixture of propellant and solvent, or when a co-solvent is not desirable. Anti-asthmatic drugs, steroids, and antibiotics are delivered as suspension aerosols. When the valve is actuated, the suspension formulation is emitted as an aerosol and the propellant rapidly vaporizes and leaves a fine dispersion of the product concentrate.

Formulation considerations for suspension aerosols, not necessary with solution aerosols, include agglomeration, particle size growth, valve clogging, moisture content, and particle size of the dispersed aerosolized particles. Lubricants such as isopropyl myristate and light mineral oil, and surfactants such as sorbitan trioleate, oleic acid, and lecithin have been used to overcome the difficulties of particle size agglomeration and growth which are directly related to the clogging problems. The moisture content of the entire formulation should be kept below 200 to 300 ppm so all of the ingredients need to be the anhydrous form of the chemical or be capable of becoming anhydrous after a drying process. The particle size of the insoluble product concentrate ingredients should be in the 1 to 10 μm range for inhalation aerosols and between 40 to 50 μm for topical aerosols.

The product concentrate in an emulsion aerosol will consist of the active ingredient, aqueous and/or nonaqueous vehicles, and a surfactant. Depending on the components, the emitted product can be a stable foam (shaving cream type) or a quick breaking foam. A quick breaking foam creates a foam when emitted from the container but the foam collapses in a relatively short time. This type of foam is used to apply the product concentrate to a large area without having to manually rub or spread the product. Also, the active drug is more rapidly available because the foam quickly collapses.

Foams are produced when the product concentrate is dispersed throughout the propellant and the propellant is in the internal phase; i.e., the emulsion behaves like o/w emulsions. When the propellant is in the external phase (i.e., like a w/o emulsion), foams are not created but sprays or wet streams result. Stable foams are produced when surfactants are used that have limited solubility in both the organic and aqueous phases. Surfactants concentrate at the interface between the propellant and the aqueous phase forming a thin film referred to as the “lamella.” It is the specific composition of this lamella that dictates the structural strength and general characteristics of the foam. Thick and tightly layered lamellae produce very structured foams which are capable of supporting their own weight. Surfactants used in emulsion aerosols have included fatty acids saponified with triethanolamine, anionic surfactants, and more recently nonionic surfactants such as the polyoxyethylene fatty esters, polyoxyethylene sorbitan esters, alkyl phenoxy ethanols, and alkanolamides. The nonionic surfactants are present fewer compatibility problems because they charge no electronic charge.

Foam technology is evidenced in the MIH Medical Library as https://pmc.ncbi.nlm.nih.gov/articles/PMC10381104/

Aqueous disinfection foams are mainly generated by mixing air with the liquid biocidal product or its application solution at a desired ratio prior to its release through a nozzle. The foam mixture is produced either actively via the injection of pressurized air into the liquid formulation or is based on the Venturi principle to mix water, a concentrated disinfection solution, and air. Non-aqueous biocidal foams, on the other hand, are generated from pressurized cans (such as rodenticides or insecticides) that contain the disinfection formulation and a blowing agent, for example, a compressed hydrocarbon forming the gas phase of the foam by expansion. Foams may be formed by the interactivity of ingredients, surface tension of ingredients, pressure release upon exposure to the ambient environment, and the action of the delivery nozzle.

As complicated as the aerosol delivery container can be, there has been discovered by Applicant the unexpected need with respect to the components of foam compositions in the invention that different propellants impact the performance of deliverables by chemical or conditional (e.g., pH) environments of the composition, both in storage and upon delivery. In particular, the oxidative and pH sensitivity (causing decomposition) of the critical ascorbate ingredient of the composition is strongly impacted by the chemical. pH and oxidative nature of the propellant. Even with protective additives in the concentrate (such as pH buffers, surfactants, antioxidants, and the like, the molecularly mobile properties of the propellant (especially as a gas) can quickly and easily cause decomposition of the ascorbate into ascorbic acid, which as explained herein can have adverse effects upon the human skin.

Antioxidants may be used with any of the known or later commercialized ascorbates, such a sodium ascorbyl phosphate, 3-glycosyl ascorbate, magnesium ascorbyl phosphate, tertrahexadecyl ascorbate, ascorbyl glucoside, ascorbyl palmitate, 3-O-ethyl ascorbic acid, PinewayC™, LiposonoIV.C, Vitamin C metabolites, 3-O-laurylglyceryl ascorbate, bis-glyceryl ascorbate, aminopropyl ascorbyl phosphate, magnesium ascorbate, calcium ascorbate, and the like.

What is claimed is:

1. An aerosol delivery system for the delivery of an aqueous composition comprising water and an oil-soluble bis-ascorbate in a weight ratio of about 0.1 to 25% by weight of the total aqueous composition, wherein the system comprises a) a housing, an actuator, an aqueous composition delivery stem, a nozzle, and a dip tube extending into a storage compartment within the housing, and further wherein the storage compartment oxygen-free and contains the aqueous composition and a propellant having a pH of greater than 4.7 when dissolved in distilled water at 20 °C and 1 atmosphere.

2. The aerosol delivery system of claim 1 wherein the propellant is selected from the group consisting of hydrofluoroolefins, nitrogen, and nitrogen oxides.

3. The system of claim 2 wherein the aqueous composition is at a pH of 5.5-8.0, and the propellant comprises 5-30% by weight of the aqueous composition within the housing.

4. The system of claim 3 wherein the aqueous comprises water, a surfactant, and bisglyceryl ascorbate at a pH of 6.5-7.8.

5. The system of claim 3 wherein the aqueous composition comprises water, less than 0.5% by weight ascorbic acid, surfactant, pH buffering agent for a pH of 7.0-7.5, skin conditioner and bisglyceryl ascorbate.

6. The system of claim 5 wherein the propellant comprises trans-1,3,3,3-Tetrafluoroprop-1-ene.

7. The system of claim 6 wherein a foaming agent having a pH of greater than 6.5 is present in the aqueous composition.

8. The system of claim 2, wherein the nozzle is configured to deliver the aqueous composition as a foam.

9. The system of claim 5, wherein the nozzle is configured to deliver the aqueous composition as a foam.

10. A method of dehalogenating skin, hair and/or nails of humans comprising contacting said skin, hair and/or nails with delivery system of claim 2 by using the actuator to release the aqueous composition as a foam, applying the foam to skin, hair and/or nails of a human, and allowing the applied foam sufficient time to reduce halogen effects on the skin, hair and/or nails.

11. A method of dehalogenating skin, hair and/or nails of humans comprising contacting said skin, hair and/or nails with delivery system of claim 3 by using the actuator to release the aqueous composition as a foam, applying the foam to skin, hair and/or nails of a human, and allowing the applied foam sufficient time to reduce halogen effects on the skin, hair and/or nails.

12. A method of dehalogenating skin, hair and/or nails of humans comprising contacting said skin, hair and/or nails with delivery system of claim 5 by using the actuator to release the aqueous composition as a foam, applying the foam to skin, hair and/or nails of a human, and allowing the applied foam sufficient time to reduce halogen effects on the skin, hair and/or nails.

13. A method of dehalogenating skin, hair and/or nails of humans comprising contacting said skin, hair and/or nails with delivery system of claim 6 by using the actuator to release the aqueous composition as a foam, applying the foam to skin, hair and/or nails of a human, and allowing the applied foam sufficient time to reduce halogen effects on the skin, hair and/or nails.

14. The method of claim 9 wherein efficiency the method has results determined on human hair having an average thickness of from 50-100 micrometers is immersed in chlorinated water having 2 ppm free chlorine and less than 0.2 ppm combined chlorine for 15 minutes and this hair after immersion is then washed for 1.5-2.0 minutes with the aqueous composition and rinsed in tap water with scrubbing to remove at least 95% by weight of the applied composition, and then the rinsed hair is leached in 1.0 liters of deionized water for 2 minutes and the free chlorine level and the combined chlorine level is measured as the free chlorine level is below 0.1 ppm and the combined chlorine level is below 0.05 ppm.

15. The method of claim 10 wherein efficiency the method has results determined on human hair having an average thickness of from 50-100 micrometers is immersed in chlorinated water having 2 ppm free chlorine and less than 0.2 ppm combined chlorine for 15 minutes and this hair after immersion is then washed for 1.5-2.0 minutes with the aqueous composition and rinsed in tap water with scrubbing to remove at least 95% by weight of the applied composition, and then the rinsed hair is leached in 1.0 liters of deionized water for 2 minutes and the free chlorine level and the combined chlorine level is measured as the free chlorine level is below 0.1 ppm and the combined chlorine level is below 0.05 ppm.

16. The method of claim 11 wherein efficiency the method has results determined on human hair having an average thickness of from 50-100 micrometers is immersed in chlorinated water having 2 ppm free chlorine and less than 0.2 ppm combined chlorine for 15 minutes and this hair after immersion is then washed for 1.5-2.0 minutes with the aqueous composition and rinsed in tap water with scrubbing to remove at least 95% by weight of the applied composition, and then the rinsed hair is leached in 1.0 liters of deionized water for 2 minutes and the free chlorine level and the combined chlorine level is measured as the free chlorine level is below 0.1 ppm and the combined chlorine level is below 0.05 ppm.

17. A solution aerosol as a two phase system comprising an oxygen-free product concentrate in a propellant at a pH of 5.0-8.0, wherein the oxygen-free product concentrate comprises water and an oil-soluble bis-ascorbate in a weight ratio of about 0.1 to 25% by weight of the total aqueous composition, and the propellant has a pH of greater than 4.7 when dissolved in distilled water at 20 °C and 1 atmosphere.

18. The solution aerosol of claim 17 wherein the propellant is selected from the group consisting of hydrofluoroolefins, nitrogen, and nitrogen oxides.

19. The solution aerosol of claim 18 wherein the aqueous composition comprises water, less than 0.5% by weight ascorbic acid, surfactant, pH buffering agent for a pH of 7.0-7.5, skin conditioner and bisglyceryl ascorbate.

20. The system of claim 4 wherein the propellant comprises 10-25% by weight of the aqueous composition within the housing.
